(19) United States
(12) Patent Application Publication
(10) Pub. No.: US 2011/0015416 A1
(43) Pub. Date: Jan. 20, 2011

(54) METHOD FOR MIXING FLUIDS, METHOD FOR PRODUCING PARTICULATES, AND PARTICULATES

(75) Inventors: Tomohide UEYAMA, Ashigarakami-gun (JP); Hideharu NAGASAWA, Ashigarakami-gun (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM CORPORATION, Tokyo (JP)

(21) Appl. No.: 12/834,634

(22) Filed: Jul. 12, 2010

(30) Foreign Application Priority Data
Jul. 17, 2009 (JP) 2009-169315

(51) Int. Cl. B01J 19/06; C07C 233/01; C11C 3/00

(52) U.S. Cl. 554/63; 264/9; 554/35; 554/61; 137/7

(57) ABSTRACT

A method for mixing fluids that can control mixing characteristics of fluids, a method for producing particulates that can provide desired particulates, and particulates produced thereby are provided. According to an embodiment of the present invention, in a method for mixing at least two kinds of fluids in a microreactor, a first fluid is supplied to a mixing zone through a divided supply passage, and a second fluid is supplied to the mixing zone through a supply passage. A total value of dynamic pressures of the first and second fluids is controlled to mix the first and second fluids in the mixing zone. Then, the mixed fluids are flowed into a micro passage.

### BACKGROUND OF THE INVENTION

1. Field of the Invention

The present invention relates to a method for mixing fluids, a method for producing particulates, and particulates, and particularly relates to a technique for mixing several kinds of fluids using a microreactor.

2. Description of the Related Art

In recent years, a technique for mixing and react several kinds of fluids with each other to form particulates by using a microreactor has been proposed. For example, WO 00/62913 describes a method for increasing a molecular diffusion velocity by dividing each fluid into fine segments and contacting the fine segments at an interface thereof as a method for accelerating mixing.

Application of the method described in WO 00/62913 to formation of particulates can control a particle size. On the other hand, unfortunately, use of the method in a fast reaction undesirably causes clogging due to precipitation.

Japanese Patent Application Laid-Open No. 2005-288254 describes a method for forcibly increasing a contact area by external or internal energy, for example. Japanese Patent Application Laid-Open No. 2005-288254 describes a method for mixing fluids in which a plurality of fluids supplied to a microdevice are merged so as to intersect at one point of a central axis of a subchannel, thereby enabling uniform and instant mixing.

In the method described in Japanese Patent Application Laid-Open No. 2005-288254, it is known that the internal energy is desirably increased by increasing flow rates of the fluids to be mixed. Unfortunately, mixing conditions in order to obtain target particulates are not disclosed specifically.

### SUMMARY OF THE INVENTION

The present invention has been made in consideration of such circumstances. An object of the present invention is to provide a method for mixing fluids that can control mixing characteristics of the fluids without clogging due to precipitation, a method for producing particulates that can produce desired particulates, and particulates produced thereby.

In order to achieve the object, a method for mixing fluids according to the present invention is a method for mixing at least two kinds of fluids in a microreactor, the method comprising the steps of: supplying a first fluid to a mixing zone through a first passage; supplying a second fluid to the mixing zone through a second passage; controlling a total value of dynamic pressures of the first and second fluids and merging the first and second fluids in the mixing zone; and flowing the first and second fluids mixed in the mixing zone into a third passage.

The inventors carefully observed a method for mixing fluids in which particulates are formed by colliding several kinds of fluids to mix the fluids. As a result, the inventors found out that mixing characteristics of the fluids can be controlled by controlling a total value of dynamic pressures determined from flow rates in passages, and achieved the present invention.

Here, the dynamic pressure refers to kinetic energy of a fluid per unit volume, and means a value determined by (½)ρV² (ρ: a density of the fluid [kg/m³], and V: a velocity of the fluid that goes to a merging point [m/s]). Mixing of the fluids includes mixing accompanied by a reaction.

In an aspect of the method for mixing fluids according to the present invention, the total value of the dynamic pressures of the first and second fluids is preferably not less than 100 kPa and not more than 10 MPa. In the aspect of the method for mixing fluids according to the present invention, the total value of the dynamic pressures of the first and second fluids is more preferably not less than 200 kPa and not more than 3 MPa. According to the present invention, mixing can be performed more quickly at the total value of the dynamic pressures of the first and second fluids of preferably not less than 100 kPa and not more than 10 MPa, and more preferably not less than 200 kPa and not more than 3 MPa. Use of this mixing method can provide fine and monodisperse particulates.

In the aspect of the method for mixing fluids according to the present invention, a volume of the mixing zone is preferably 0.001 mm³ to 1 mm³, and more preferably 0.005 to 0.5 mm³. The volume of the mixing zone less than 0.001 mm³ makes pressure loss too large to flow the fluids at a realistic flow rate for production of particulates. The volume of the mixing zone more than 1 mm³ causes distribution in the mixing state in the mixing zone so that distribution of the particulates to be produced may be polydisperse.

In the aspect of the method for mixing fluids according to the present invention, a total flow rate per second supplied to the mixing zone is preferably 1×10³ to 1×10⁵ times the volume of the mixing zone, and more preferably 5×10³ to 5×10⁴ times. It is because the dynamic pressure may not reach 100 kPa at a volume of the total flow rate less than 1×10³ times the volume of the mixing zone, and because pressure loss may be large at a volume of the total flow rate more than 1×10⁵ times the volume of the mixing zone.

In the aspect of the method for mixing fluids according to the present invention, preferably, at least one passage of the first passage and the second passage includes a plurality of subpassages branched halfway to the mixing zone, and a central axis of one fluid that flows through at least one subpassage of the plurality of subpassages intersects with a central axis of other fluid that flows through other passage at one point in the mixing zone.

According to the present invention, before two or more kinds of fluids are merged, at least one fluid is divided into a plurality of fluids. Then, the two or more kinds of fluids are merged so that the central axis of at least one divided fluid may intersect with the central axis of the other fluid at one point of the mixing zone at a predetermined intersection angle. When these divided flows merge and collide, the flows are instantaneously divided into smaller fluid lumps by the kinetic energy that the flows have. Thereby, a contact area of the fluids can be increased and a diffusion mixing distance can be reduced to achieve instant mixing.

Here, the central axis of the fluid means a center line in a cylindrical axis direction when the fluid flowing in the passage has a cylindrical shape, for example. When the fluid flowing in the passage has a shape other than a cylindrical shape, the central axis corresponds to an axis in a length direction of the passage that passes through a center of gravity (geometric center of gravity) of a cross section perpendicular to the length direction of the passage.

In the aspect of the method for mixing fluids according to the present invention, a diameter of the third passage is preferably not more than an equivalent diameter of a merging passage.

According to the present invention, the third passage having the diameter not more than the equivalent diameter of the merging passage can prevent two fluids from not colliding, in other words, the two fluids from flowing into an outlet before the two fluids merge and collide at one point. As a result, a dynamic pressure as kinetic energy per unit volume can be more effectively given to the fluid.

In order to achieve the object, in a method for producing particulates according to the present invention, a first fluid is mixed with a second fluid by one of the above-mentioned methods for mixing fluids, the first fluid being a solution obtained by dissolving an organic compound in a good solvent having relatively high solubility for the organic compound, and the second fluid being a solution obtained by dissolving the organic compound in a poor solvent having relatively low solubility for the organic compound.

According to the present invention, particulates having a small particle size and high monodispersity can be produced.

In the aspect, a ratio of the dynamic pressure of the first fluid to that of the second fluid is preferably 1:0.1 to 1:100. By mixing so that the dynamic pressure ratio may be 1:0.1 to 1:100, from the viewpoint of productivity, it is possible to prevent the organic compound deposited in an excessively high concentration from aggregating, and it is possible to prepare the first fluid and the second fluid in such a range that concentrations of the first fluid and the second fluid are not excessively low. From the viewpoint of productivity and stabilization of the flow, the dynamic pressure ratio 1:1 is most preferable.

In the aspect of the method for producing particulates according to the present invention, the organic compound is preferably a material insoluble or sparingly soluble in an aqueous medium.

Here, insolubility in an aqueous medium means that solubility in 100 mL of the aqueous medium is not more than 0.001 g at 25° C. Sparing solubility in an aqueous medium means that solubility in 100 mL of the aqueous medium is from 0.001 to 1 g at 25° C.

In order to achieve the object, particulates according to the present invention are produced by the above-mentioned method for producing particulates.

In the present invention, a “microreactor” generally refers to an apparatus for flowing and merging fluids in a fine passage and performing operation such as mixing, a reaction, and heat exchange attributed to flowing and merging the fluids. A diameter or equivalent diameter of the fine passage or the flow flowing in the fine passage is not more than 1 mm, and the diameter or equivalent diameter thereof is particularly not more than 500 μm. The equivalent diameter is used in a sense used in hydrodynamics.

According to the method for mixing fluids according to the present invention, the mixing characteristics of the fluids can be controlled by controlling the total value of the dynamic pressures of the first and second fluids. The mixing characteristics of the fluids can be improved by controlling the total value of the dynamic pressures at a value of not less than a predetermined value. According to the method for producing particulates according to the present invention, fine and monodisperse particulates without clogging can be produced.

### BRIEF DESCRIPTION OF THE DRAWINGS

FIG. 1 is a conceptual diagram of a flat microreactor used in the present invention;

FIG. 2 is a conceptual diagram showing a modification of the flat microreactor used in the present invention;

FIG. 3 is a conceptual diagram showing other modification of the flat microreactor used in the present invention;

FIG. 4 is a conceptual diagram of a three-dimensional microreactor used in the present invention;

FIGS. 5A and 5C are plan views of the three-dimensional microreactor and FIG. 5B is a drawing observed from a cross section thereof;

FIGS. 6A and 6B are conceptual diagrams of a flat T-shaped microreactor used in the present invention;

FIGS. 7A and 7B are conceptual diagrams of a flat Y-shaped microreactor used in the present invention;

FIG. 8 is a conceptual diagram showing a mixing zone of other T-shaped microreactor;

FIGS. 9A and 9B are graphs showing a relationship between a total dynamic pressure and a particle size d50;

FIGS. 10A and 10B are graphs showing a relationship between a total dynamic pressure and distribution Mv/Mn; and

FIG. 11 is a configuration diagram of a fluid mixing system.

### DETAILED DESCRIPTION OF THE PREFERRED EMBODIMENTS

Hereinafter, preferable embodiments according to the present invention will be described with reference to the accompanying drawings. While the present invention will be described by the following preferable embodiments, modification can be made by various methods and embodiments other than the described embodiments can be used without deviating from the scope of the present invention. Accordingly, all the modifications within the scope of the present invention are included in the scope of claims. A range of a numerical value expressed using “to” herein means a range including the values written before and after “to.”

A method for mixing fluids according to the present invention is a method for mixing at least two kinds of fluids in a microreactor, the method comprising the steps of: supplying a first fluid to a mixing zone through a first passage; supplying a second fluid to the mixing zone through a second passage; controlling a total value of dynamic pressures of the first and second fluids and merging the first and second fluids in the mixing zone; and flowing the first and second fluids mixed in the mixing zone into a third passage.

In the method for mixing fluids in which particulates are formed by mixing several kinds of fluids by collision, mixing characteristics of the fluids can be controlled by controlling a total value of dynamic pressures determined from flow rates of the respective passages. Use of this method for mixing fluids can provide particles having a desired particle size.

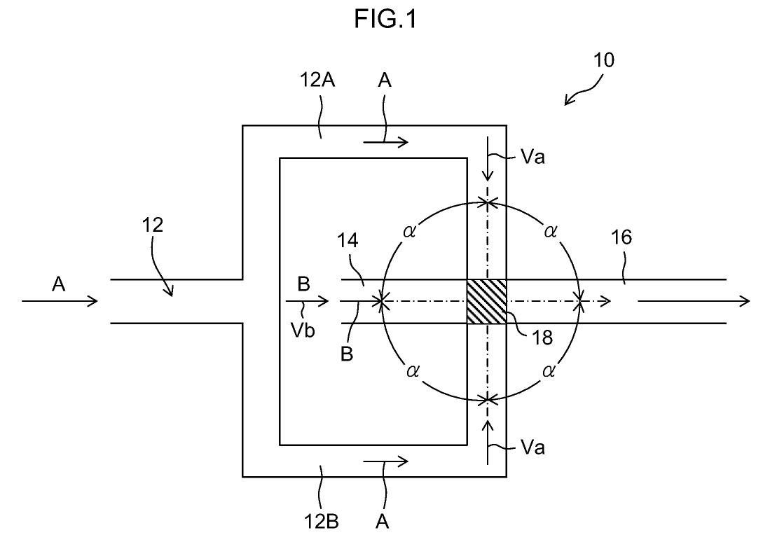

FIG. 1 shows an example of a microreactor used in order to mix at least two kinds of fluids. As shown in FIG. 1, a microreactor 10 includes two divided supply passages 12A and 12B that are branched from halfway of one supply passage 12 that supplies a first fluid A, and can divide the first fluid A into two. The microreactor 10 also includes one supply passage 14 that supplies a second fluid B and is not divided, and a micro passage 16 in which the first fluid A is reacted with the second fluid B and the reacted fluid is flowed. The microreactor 10 is formed so that the divided supply passages 12A and 12B, the supply passage 14, and the micro passage 16 may communicate with each other in one mixing zone 18. The divided supply passages 12A and 12B, the supply passage 14, and the micro passage 16 are disposed every 90° around the mixing zone 18 within substantially the same plane. Namely, central axes (dashed dotted lines) of the respective passages 12A, 12B, 14, and 16 intersect in a cross shape (intersection angle of α=90°) in the mixing zone 18. While only the supply passage 12 for the first fluid A is divided in FIG. 1, the supply passage 14 for the second fluid B may also be divided into several passages. The intersection angle α of the respective passages 12A, 12B, 14, and 16 disposed around the mixing zone 18 can be set properly at an angle other than 90° without limitation. The number of division of the supply passages 12 and 14 is not limited in particular, and preferably 2 to 10 and more preferably 2 to 5 because an excessively large number of the divided supply passages complicates the structure of the microreactor 10.

Next, a method for determining a total value of dynamic pressures will be described on the basis of the microreactor 10 in FIG. 1. In order to determine the total value of the dynamic pressures, the dynamic pressures of the fluids that flow in the respective passages 12A, 12B, and 14 are determined. When a density of the fluid A is pa [kg/m³] and a velocity of the fluid A that goes to the mixing zone 18 is Va [m/s], the dynamic pressure Pda of the fluid A is determined by the following equation:

Pda=(½)×ρa×Va²×2(the number of passages). (1)

When a density of the fluid B is ρb [kg/m³] and a velocity of the fluid B that goes to the mixing zone 18 is Vb [m/s], the dynamic pressure Pdb of the fluid B is determined by the following equation:

Pdb=(½)×ρb×Vb²×1(the number of passages). (2)

Then, the total value Pto of the dynamic pressures of the respective fluids is Pto=Pda+Pdb. A velocity of each fluid can be determined by dividing a flow rate per unit time [m³/s] of each fluid flowing each passage by a cross-section area [m²] of each passage.

In the above-mentioned example, a case where the fluid A has two passages and the fluid B has one passage has been described. The total value of the dynamic pressures can also be determined similarly when not less than three kinds of fluids are contained and when each passage branches into two or more.

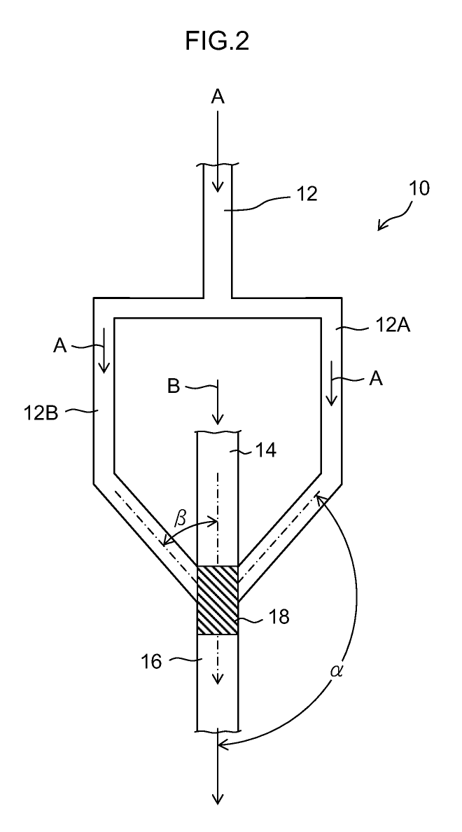

FIG. 2 shows a modification of the flat microreactor 10 in FIG. 1. An intersection angle β that the central axis of the divided supply passages 12A and 12B makes with the central axis of the supply passage 14 is 45° and formed smaller than 90° in the case of FIG. 1. The microreactor 10 in FIG. 2 is formed so that the intersection angle α that the central axis of the micro passage 16 makes with the central axis of the divided supply passages 12A and 12B is 135°.

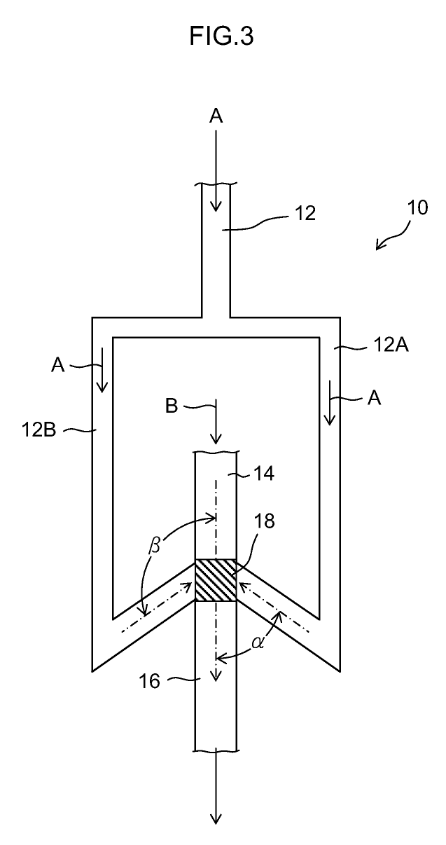

FIG. 3 shows other modification of the flat microreactor 10 in FIG. 1. The intersection angle β that the central axis of the divided supply passages 12A and 12B through which the first fluid A flows makes with the central axis of the supply passage 14 through which the second fluid B flows is 135° and formed larger than 90° in the case of FIG. 1. The microreactor 10 in FIG. 3 is formed so that the intersection angle α that the central axis of the micro passage 16 makes with the central axis of the divided supply passages 12A and 12B is 45°. The intersection angle α that the supply passage 14 makes with the divided supply passages 12A and 12B, the intersection angle β that the divided supply passages 12A and 12B make with the micro passage 16, and the cross-section areas of the passages 12A, 12B, 14, and 16 can be set depending on specific conditions. However, it is preferable that the intersection angles α and β and the cross-section areas of the passages 12A, 12B, 14, and 16 are set so as to satisfy S1>S2 when S1 is a total of the cross-section areas in a thickness direction of all the merged solution of the second fluid B and the first fluid A, and S2 is the cross-section area in a diameter direction of the micro passage 16. Thereby, the contact area between the fluids A and B is further increased, and the diffusion mixing distance can be further reduced so that it is easier to provide instant mixing.

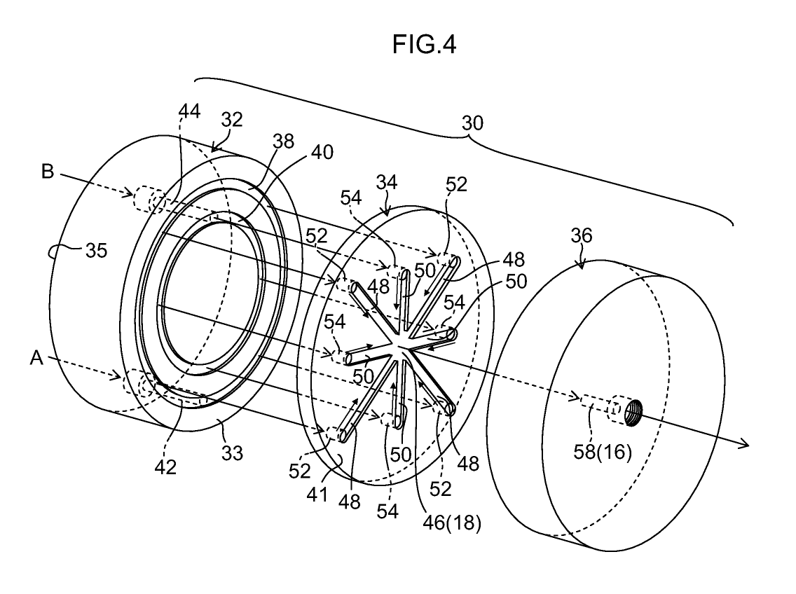

FIG. 4 shows an example of a three-dimensional microreactor 30 used in order to mix at least two kinds of fluids, and is an exploded perspective view showing that three parts that constitute the microreactor 30 are disassembled. Same reference numerals will be given to portions having the same function as those in FIGS. 1 to 3 and described.

The three-dimensional microreactor 30 is mainly formed of a supply block 32, a merging block 34, and a reaction block 36 each having a cylindrical shape. Then, in order to assemble the microreactor 30, side surfaces of these blocks 32, 34, and 36 having a cylindrical shape are disposed in this order so as to have a cylindrical shape. In this state, the blocks 32, 34, and 36 are integrally fixed with bolts and nuts, for example.

Two annular grooves 38 and 40 are concentrically provided on a side surface 33 of the supply block 32 facing the merging block 34. In the state where the microreactor 30 is assembled, the two annular grooves 38 and 40 form ring-shaped passages through which the second fluid B and the first fluid A flow, respectively. Then, through holes 42 and 44 that respectively reach the outer annular groove 38 and the inner annular groove 40 are formed from a side surface 35 of the supply block 32 on the opposite side, which does not face the merging block 34. Among the two through holes 42 and 44, a supplying device (e.g. a pump, a connecting tube) that supplies the first fluid A is connected with the through hole 42 that communicates with the outer annular groove 38. A supplying device that supplies the second fluid B is connected with the through hole 44 that communicates with the inner annular groove 40. While the first fluid A is flowed to the outer annular groove 38 and the second fluid B is flowed to the inner annular groove 40 in FIG. 4, the first fluid A may be flowed to the inner annular groove 40 and the second fluid B may be flowed to the outer annular groove 38.

A circular mixing zone 46 is formed in a center of a side surface 41 of the merging block 34 facing the reaction block 36. Four long radial grooves 48 and 48 . . . and four short radial grooves 50 and 50 . . . are alternately provided radially from this mixing zone 46. In the state where the microreactor 30 is assembled, the mixing zone 46 and the radial grooves 48 and 50 form a circular space serving as the mixing zone 18 and radial passages through which the fluids A and B flow. Among the eight radial grooves 48 and 50, through holes 52 and 52 . . . are respectively formed from ends of the long radial grooves 48 in a thickness direction of the merging block 34. These through holes 52 communicate with the above-described outer annular groove 38 formed in the supply block 32. Similarly, through holes 54 and 54 . . . are respectively formed from ends of the short radial grooves 50 in the thickness direction of the merging block 34. These through holes 54 communicate with the inner annular groove 40 formed in the supply block 32.

One through hole 58 that communicates with the mixing zone 46 in a thickness direction of the reaction block 36 is formed in the center of the reaction block 36, and this through hole 58 serves as the micro passage 16.

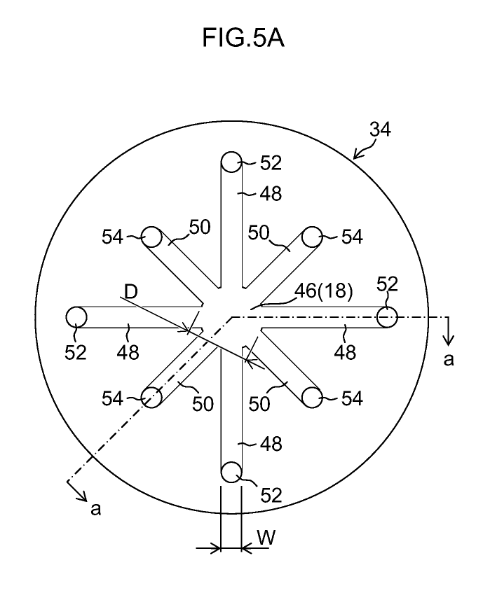

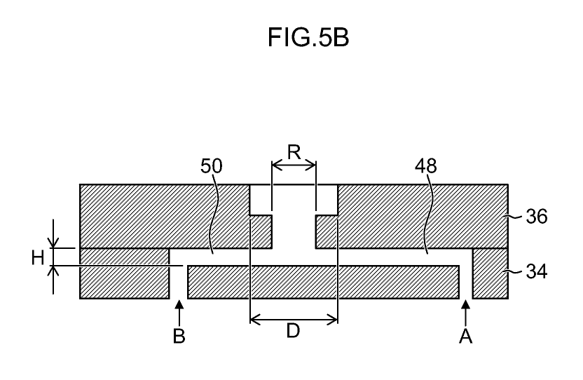

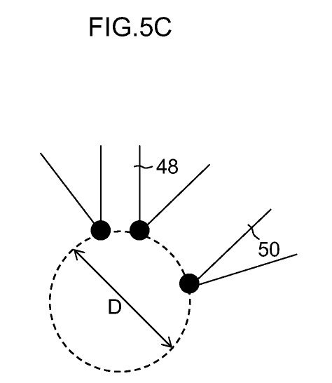

Thereby, the fluid A flows through the supply passage 12 formed of the through hole 42 of the supply block 32, the outer annular groove 38, the through hole 52 of the merging block 34, and the long radial groove 48, is divided into four divided flows, and reaches the mixing zone 18 (mixing zone 46). On the other hand, the fluid B flows through the supply passage 14 formed of the through hole 44 of the supply block 32, the inner annular groove 40, the through hole 54 of the merging block 34, and the short radial groove 50, is divided into four divided flows, and reaches the mixing zone 18 (mixing zone 46). In the mixing zone 18, the divided flows of the fluid A and the divided flows of the fluid B each have kinetic energy, and are merged, collided and mixed. Subsequently, the mixed flow changes the flow direction by 90° and flows into the micro passage 16. The fluids A and B react with each other and flow in the micro passage 16. FIG. 5A is a plan view of the merging block 34, and FIG. 5B is a sectional view taken along an a-a line of FIG. 5A. In FIG. 5, reference character W designates a width of the divided supply passages 12 and 14, reference character H designates a depth of the divided supply passages 12 and 14, reference character D designates a merging section equivalent diameter of the mixing zone 18, and reference character R designates a diameter of the micro passage 16. Usually, the merging section equivalent diameter of the mixing zone 18 is designed larger than the diameter of the micro passage 16. The merging section equivalent diameter of the mixing zone 18 may be the same as the diameter of the micro passage 16. Here, the merging section equivalent diameter means a diameter of a circle formed by connecting walls that separate the respective passages (e.g., the long radial grooves 50, the short radial grooves 48), as shown in FIG. 5C.

When a figure formed by connecting peaks of the walls, which are located on the center side of the microreactor and separate the respective passages, is not a circle, the equivalent diameter of the mixing zone is a diameter of a circle determined from the peaks by the method of least squares.

The microreactors 10 and 30 thus configured in FIGS. 1 to 4 can be produced using semiconductor processing techniques, particularly, precision machining techniques such as etching (e.g., photolitho-etching) processing, superfine electrical discharge machining, stereolithography, mirror finishing, and diffused junction techniques. Common machining techniques using a lathe or a drilling machine can also be used.

A material for the microreactors 10 and 30 is not limited in particular, and any material may be used as long as the above-mentioned processing techniques can be applied to the material. Specifically, metal materials (e.g., iron, aluminum, stainless steel, titanium, various kinds of metals), resin materials (e.g., fluorocarbon polymer, acrylic resin), and glasses (e.g., silicon, quartz) can be used.

Next, a method for mixing fluids according to the present invention using the microreactors 10 and 30 thus configured will be described. When the fluids A and B are mixed by the microreactors 10 and 30 thus configured in FIGS. 1 to 4, the two kinds of fluids are mixed through a dividing step and a merging step in both the cases of the microreactors 10 and 30. At the dividing step (supply block), at least one fluid of the two kinds of the fluids A and B is divided into several fluids. At the merging step (merging block), the two kinds of the fluids A and B are merged so that a central axis of at least one divided solution of the several divided solutions may intersect with a central axis of other solution of the two kinds of the fluids A and B at one point in the mixing zone 18. The mixing characteristics can be controlled in the mixing zone 18 by controlling the total value of the dynamic pressures of the respective fluids. The mixing characteristics particularly improve at the total value of the dynamic pressures of not less than 100 kPa and preferably not less than 200 kPa. A lower total value of the dynamic pressures reduces more energy supplied to the microreactors 10 and 30. Accordingly, the mixing characteristics of the fluids and the energy consumed can be controlled by controlling the total value of the dynamic pressures of the respective fluids.

A volume of the mixing zone in the three-dimensional microreactor is calculated by multiplying an area of a circle determined from the merging section equivalent diameter D by the depth H of the supply passage. The volume of the mixing zone is preferably 0.001 mm³ to 1 mm³, and more preferably 0.005 to 0.5 mm³. The volume of the mixing zone less than 0.001 mm³ makes pressure loss too large to flow the fluids at a realistic flow rate for production of particulates. The volume of the mixing zone more than 1 mm³ causes distribution in the mixing state in the mixing zone so that distribution of the particulates to be produced may be polydisperse.

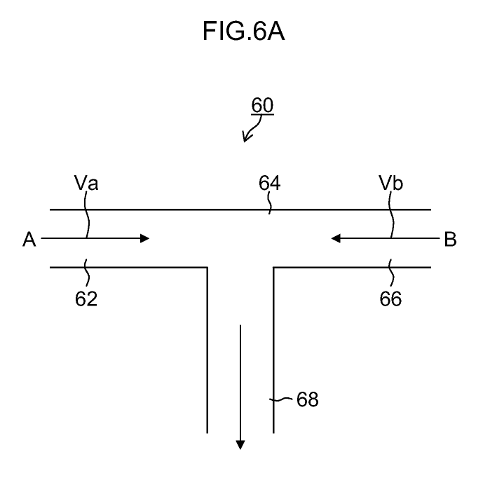

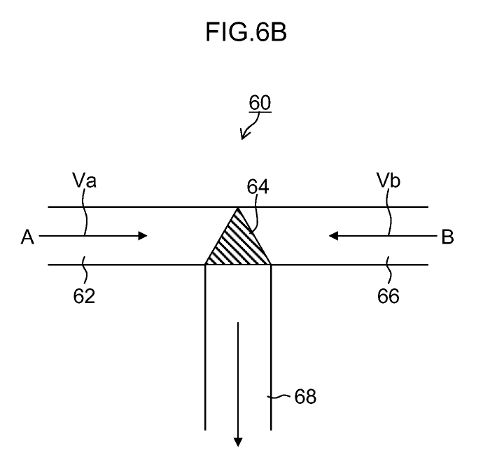

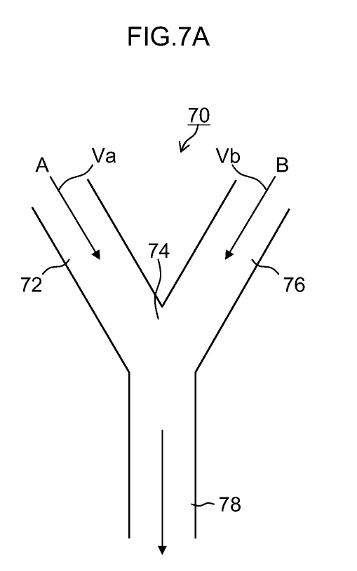

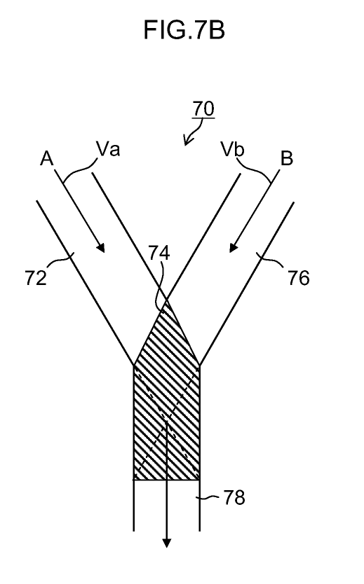

FIGS. 6 and 7 are respectively conceptual diagrams showing a structure of one aspect of a T-shaped microreactor 60 and that of a Y-shaped microreactor 70. The T-shaped microreactor 60 in FIG. 6A includes a supply passage 62 that supplies the first fluid A, a supply passage 66 that supplies the second fluid B, and a micro passage 68 in which the first fluid A is reacted with the second fluid B. The T-shaped microreactor 60 is formed so that the supply passage 62, the supply passage 66, and the micro passage 68 may communicate with each other in one mixing zone 64.

The Y-shaped microreactor 70 in FIG. 7A includes a supply passage 72 that supplies the first fluid A, a supply passage 76 that supplies the second fluid B, and a micro passage 78 in which the first fluid A is reacted with the second fluid B. The Y-shaped microreactor 70 is formed so that the supply passage 72, the supply passage 76, and the micro passage 78 may communicate with each other in one mixing zone 74.

In the mixing zone 64 of the T-shaped microreactor 60, the first fluid A at a density ρa [kg/m³] and a velocity Va [m/s] collides with the second fluid B at a density ρb [kg/m³] and a velocity Vb [m/s] to be mixed. Similarly, in the mixing zone 74 of the Y-shaped microreactor 70, the first fluid A at a density ρa [kg/m³] and a velocity Va [m/s] collides with the second fluid B at a density ρb [kg/m³] and a velocity Vb [m/s] to be mixed. The fluids A and B mixed and reacted with each other by energy of collision flow into the micro passages 68 and 78. In the micro passages 68 and 78, the fluids A and B are mixed, reacted, and flowed.

Also in the case where the two kinds of fluids are mixed using the T-shaped microreactor 60 and the Y-shaped microreactor 70, the mixing characteristics of the fluids can be controlled by controlling the total value of the dynamic pressures of the respective fluids. In the T-shaped microreactor 60 and the Y-shaped microreactor 70, the total value of the dynamic pressures is determined as follows:

Pda=(½)×ρa×Va²×1(the number of the passages); and (1)

Pdb=(½)×ρb×Vb²×1(the number of the passages). (2)

Then, the total value Pto of the dynamic pressures of the respective fluids is Pto=Pda+Pdb. The velocity of each fluid that goes to the mixing zone can be determined by dividing a flow rate per unit time of each fluid flowing [m³/s] by a cross-section area of each passage [m²].

In the T-shaped microreactor 60 and the Y-shaped microreactor 70, a merging passage equivalent diameter means a diameter of a larger passage of the two supply passages.

In the T-shaped microreactor 60 and the Y-shaped microreactor 70, a volume of the mixing zone can be determined as follows.

FIG. 6B is a conceptual diagram showing the mixing zone of the T-shaped microreactor 60. In this microreactor 60, the supply passage 62, the supply passage 66, and the micro passage 68 have the same diameter. In this case, the mixing zone 64 is a region shown by oblique lines formed by connecting a point or a line where the supply passage 62 intersects with the supply passage 66 (a point when the passages have a cylindrical shape and a line when the passages have a rectangular shape), a point or a line where an extended line of the supply passage 62 intersects with the micro passage 68, and a point or a line where an extended line of the supply passage 66 intersects with the micro passage 68.

FIG. 7B is a conceptual diagram showing the mixing zone of the Y-shaped microreactor 70. In this microreactor 70, the supply passage 72, the supply passage 76, and the micro passage 78 have the same diameter. In this case, the mixing zone 74 is a region shown by oblique lines formed by connecting a point or a line where the supply passage 72 intersects with the supply passage 76 (a point when the passages have a cylindrical shape and a line when the passages have a rectangular shape), a point or a line where an extended line of the supply passage 72 intersects with the micro passage 78, and a point or a line where an extended line of the supply passage 76 intersects with the micro passage 78.

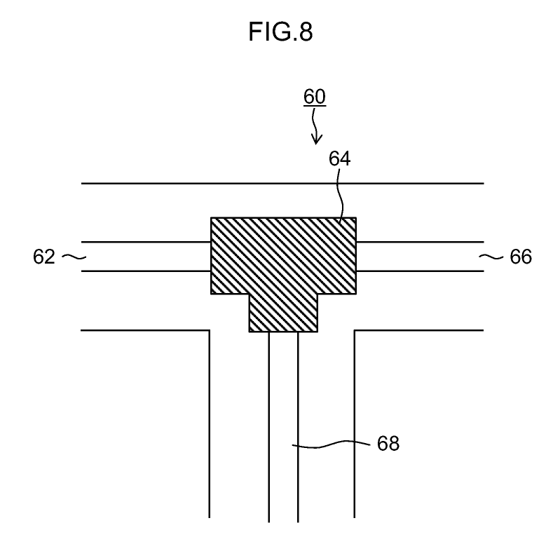

FIG. 8 shows an example of the T-shaped microreactor 60. The microreactor 60 in FIG. 8 has the supply passage 62 and the supply passage 66 whose diameter is each larger in a central portion. The passage having a larger diameter and making 90° with the supply passage 62 and the supply passage 66 communicates with the micro passage 68 having a smaller diameter.

In the T-shaped microreactor 60 in FIG. 8, an inlet passage (the supply passage 62 and the supply passage 66) and an outlet passage (micro passage 68) have a diameter different from a diameter of a region where the two solutions merge, or have a shape different from that of the region. In this case, the mixing zone 64 is a portion having a different diameter or a different shape, as shown by the oblique lines. A volume of the mixing zone 64 is a volume of the region shown by the oblique lines.

As the two kinds of the fluids used for the present invention, for example, ethanol is used as a solution obtained by dissolving an organic compound in a good solvent having relatively high solubility for the organic compound, and water is used as a poor solvent solution having relatively low solubility for the organic compound. As the particulates produced, insoluble or sparingly soluble organic compounds in an aqueous medium, particularly ceramides are used, for example.

A flowmeter, a pump, and a flow rate controller are preferably installed in a piping that supplies the fluids to above-mentioned microreactor. This allows mixing while the total of the dynamic pressures is controlled.

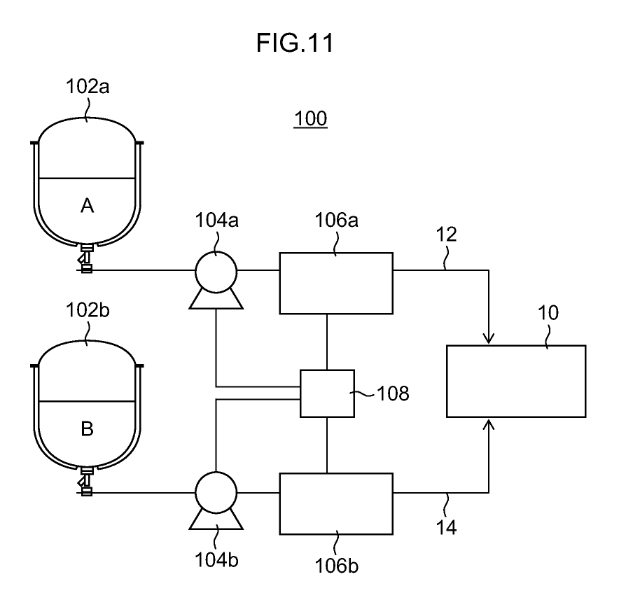

A method for mixing while controlling dynamic pressures will be described with reference to a fluid mixing system 100 in FIG. 11. The fluid mixing system 100 includes a tank 102a that stores the first fluid A, a tank 102b that stores the second fluid B, and the microreactor 10 that mixes the two fluids. The tank 102a is connected to the microreactor 10 by the supply passage 12. A pump 104a and a flowmeter 106a are installed in the supply passage 12. The tank 102b is connected to the microreactor 10 by the supply passage 14. A pump 104b and a flowmeter 106b are installed in the supply passage 14. The pumps 104a and 104b and the flowmeters 106a and 106b are electrically connected to a flow rate controller 108. On the basis of information from the flowmeters 106a and 106b, the flow rate controller 108 can control operation of the pumps 104a and 104b.

For example, when a density of the first fluid A is ρa=790 (kg/m³), a density of the second fluid B is ρb=1000 (kg/m³), the cross-section area of the first passage is Sa=2×10⁻⁸ (m³), the number of the passages is na=5, the cross-section area of the second passage is Sb=14×10⁻⁸ (m²), and the number of the passages is nb=5, a dynamic pressure ratio is 1:1, and the first fluid and the second fluid merge at the lowest necessary dynamic pressure of 100 (kPa), the lowest total flow rate Q of the fluids to be supplied to the microreactor can be determined from the following formulas:

The dynamic pressure ratio 1:1=ρa×va²/2:ρb×vb²/2 (va is a linear velocity (m/s) per passage for the first fluid A, and vb is a linear velocity (m/s) per passage for the second fluid B); (1)

The lowest necessary dynamic pressure is 100 kPa. ρa×va²/2×na+ρb×vb²/2×nb>100×10³; (2)

The total flow rate Q=Qa+Qb (Qa is a flow rate of the first liquid and Qb is a flow rate of the second fluid); (3)

Qa=va×Sa×na; and (4)

Qb=vb×Sb×nb. (5)

From (1) and (2), va=(2×100×10³/(ρa(na+nb)))^{1/2}, and vb=2×100×10³/(ρb(na+nb)))^{1/2} can be derived. From this, va=5.0 (m/s) and vb=4.5 (m/s).

From (4), Qa=5.0×2×10⁻⁸×5=5×10⁻⁷ (m³/s). From (5), Qb=4.5×14×10⁻⁸×5=3.15×10⁻⁶ (m³/s). From (3), Q=3.65×10⁻⁶ (m³/s)=219 (ml/min.). An amount of supply to the microreactor is controlled so as to exceed the lowest flow rate Q calculated.

Next, when a density of the first fluid A is ρa=790 (kg/m³), a density of the second fluid B is ρb=1000 (kg/m³), the cross-section area of the first passage is Sa=2×10⁻⁸ (m²), the number of the passages is na=5, the cross-section area of the second passage is Sb=14×10⁻⁸ (m²), and the number of the passages is nb=5, a dynamic pressure ratio is 1:1, the lowest necessary dynamic pressure is 100 (kPa), and the first fluid and the second fluid merge at the total flow rate Q=480 (ml/min)=8×10⁻⁶ (m³/s), flow rates Qa and va of the first liquid and flow rates Qb and vb of the second liquid to be supplied to the microreactor can be determined from the following formulas:

The dynamic pressure ratio 1:1=ρa×va²/2:ρb×vb²/2 (va is a linear velocity (m/s) per passage for the first fluid A, and vb is a linear velocity (m/s) per passage for the second fluid B); (1)

The lowest necessary dynamic pressure is 100 kPa. ρa×va²/2×na+ρb×vb²/2×nb>100×10³; (2)

The total flow rate Q=Qa+Qb (Qa is a flow rate of the first fluid A, and Qb is a flow rate of the second fluid B); (3)

Qa=va×Sa×na; and (4)

Qb=vb×Sb×nb. (5)

From (1), vb=(ρa/ρb)^{1/2}×va can be derived. From (3), (4), and (5), va=Q/(Sana+(ρa/ρb)^{1/2}Sbnb) can be derived. From this, va=11 m/s and vb=9.8 m/s.

From (4) and va=11 m/s, Qa=66.4 ml/min. From (5) and vb=9.8 m/s, Qb=413.6 ml/min.

### [A Ceramide Dispersed Object]

A ceramide dispersed object of the present invention contains: (1) ceramide containing particles that contain ceramides, are dispersed in an aqueous phase as an oil phase component, and have a volume average particle size of not less than 1 nm and not more than 100 nm; (2) at least one fatty acid component of (a) fatty acids and (b) fatty acid salts; (3) polyhydric alcohol having an amount of not less than 5 times and not more than 20 times an amount of the ceramides; and (4) polysaccharide fatty acid ester; and has pH of not less than 6 and not more than 8.

### (1) The Ceramide Containing Particles

The ceramide containing particles in the present invention contain ceramides, are dispersed in an aqueous phase as an oil phase component, and have a volume average particle size of not less than 1 nm and not more than 100 nm.

Ceramides in the present invention include ceramides and derivatives thereof irrespective of the origin such as synthetic products or extracted products. “Ceramides” in the present invention include natural ceramides described later, compounds having natural ceramides as a basic skeleton, and precursors that can derive these compounds, and generally refer to natural ceramides, sugar-modified ceramides such as sphingoglycolipid, ceramide analogs, sphingosines and phytosphingosines, and derivatives thereof.

### (Natural Ceramides)

In the present invention, natural ceramides mean ceramides having the same structure as that of ceramides that exist in the human skin. A more preferable aspect of natural ceramides is an aspect that includes no sphingoglycolipid and has not less than 3 hydroxyl groups in the molecular structure.

Hereinafter, natural ceramides that can be used for the present invention will be described in detail.

Examples of a basic structural formula of natural ceramides that can be used suitably for the present invention are shown in (1-1) to (1-11) below.

(1-1) is a compound known as ceramide 1, (1-2) is a compound known as ceramide 9, (1-3) is a compound known as ceramide 4, (1-4) is a compound known as ceramide 2, (1-5) is a compound known as ceramide 3, (1-6) is a compound known as ceramide 5, (1-7) is a compound known as ceramide 6, (1-8) is a compound known as ceramide 7, (1-9) is a compound known as ceramide 8, and (1-11) is a compound known as ceramide 3B.

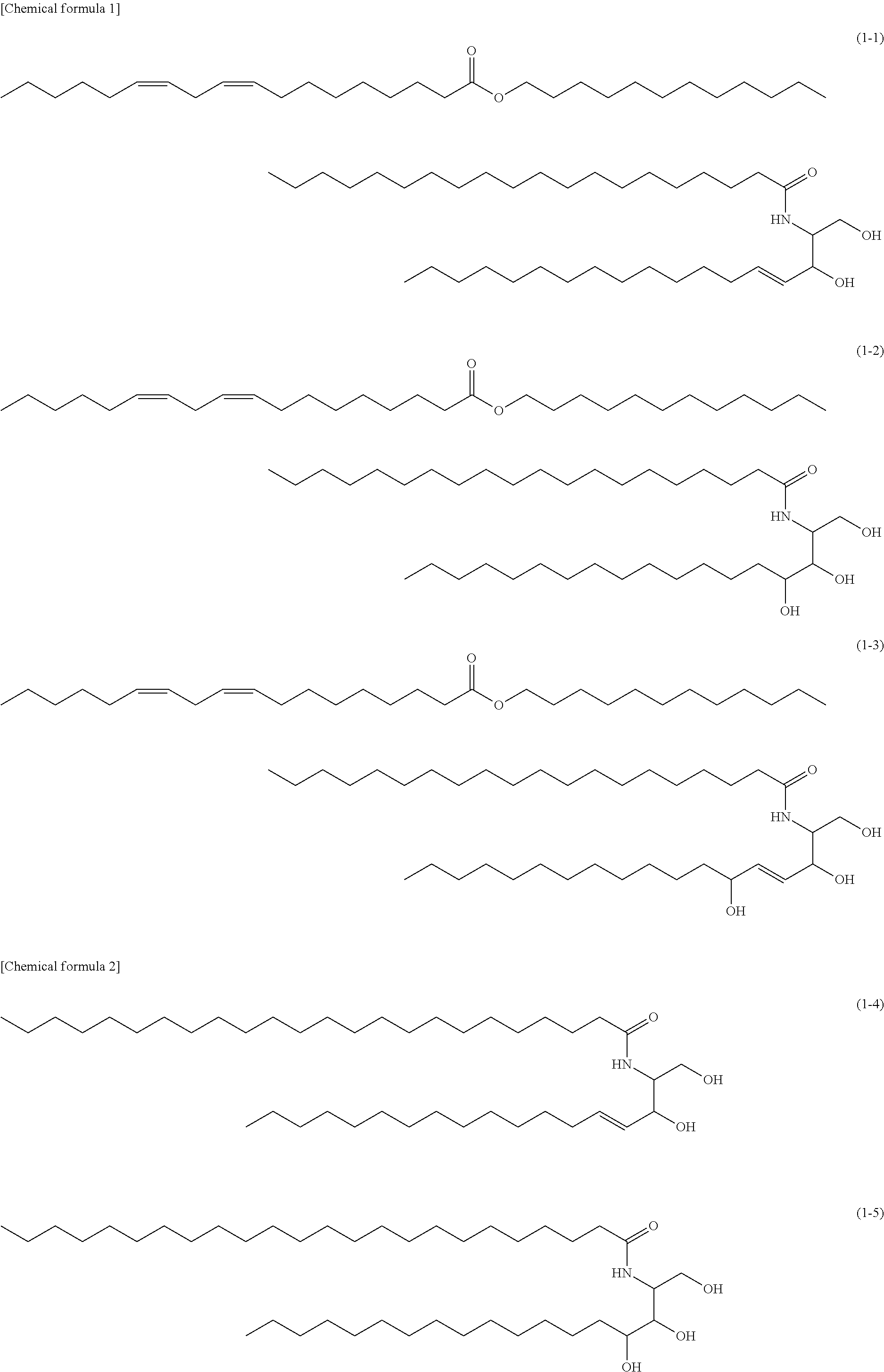

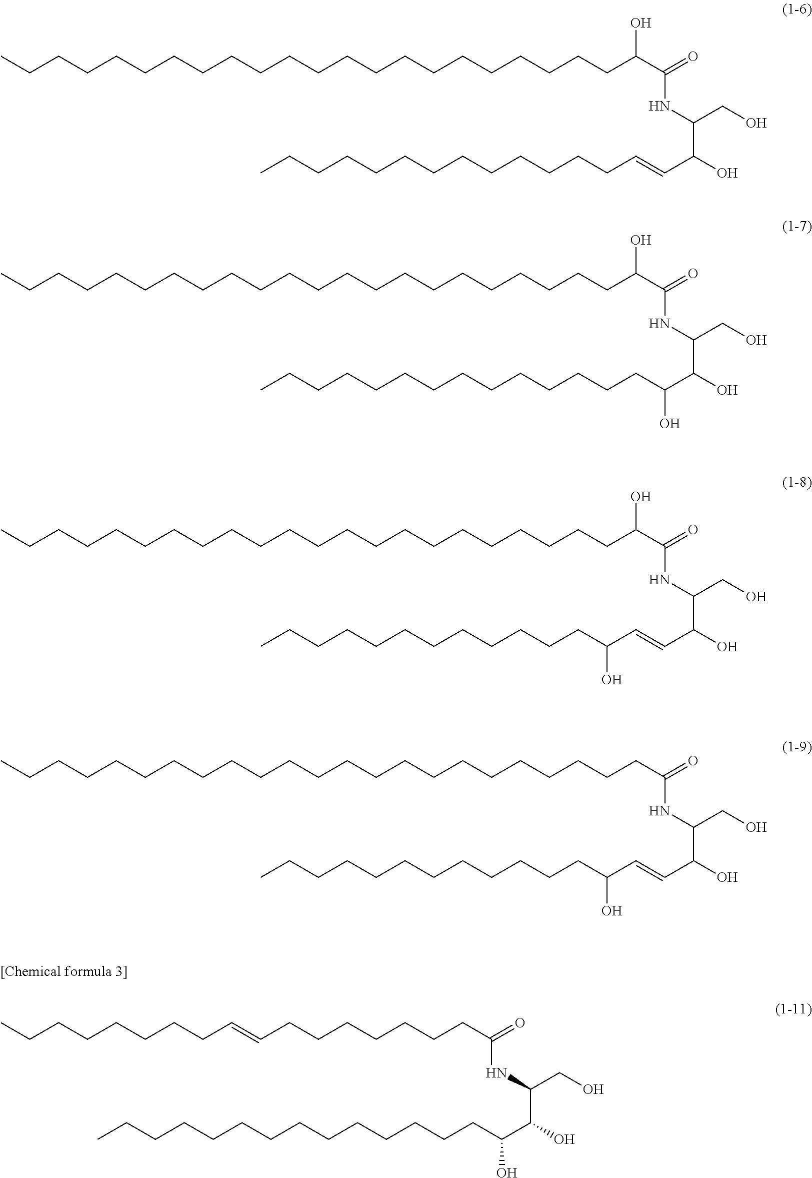

The structural formulas show an example about each ceramide. These ceramides are natural products and ceramides actually originating from human beings and animals have variation in a length of the above-mentioned alkyl chain. Accordingly, the alkyl chain length may have any structure as long as the alkyl chain has the above-mentioned skeleton.

Additionally, the ceramides modified according to a purpose, for example, introduction of a double bond in a molecule to give solubility for formulation, or introduction of hydrophobic groups to give permeability, can also be used.

These ceramides having a general structure generally referred to as a natural type are obtained from natural products (extraction) or by microbial fermentation, while these ceramides may further contain synthesized products and ceramides of animal origin.

Such natural ceramides are available also as commercial products, and examples of the natural ceramides include Ceramide I, Ceramide III, Ceramide IIIA, Ceramide IIIB, Ceramide IIIC, Ceramide VI (made by Cosmo Pharm), Ceramide TIC-001 (made by Takasago International Corporation), CERAMIDE II (made by Quest International), DS-Ceramide VI, DS-CLA-Phytoceramide, C6-Phytoceramide, DS-ceramide Y3S (made by DOOSAN Corporation), and CERAMIDE 2 (made by Sederma S.A.S.). The compound (1-5) exemplified above is available as “CERAMIDE 3” [trade name, made by Evonick Industries AG (the former Degussa AG)]. The compound (1-7) exemplified above is available as “CERAMIDE 6” [trade name, made by Evonick Industries AG (the former Degussa AG)].

Natural ceramides contained in the ceramide containing particles may be used alone, or may be used in combination with not less than two kinds. From the viewpoint of stabilization of emulsification and handling properties, natural ceramides are preferably used in combination with not less than two kinds because ceramides usually have a high melting point and high crystallinity.

A particle size of the ceramide containing particles can be measured by a commercially available particle size distribution analyzer or the like.

As a method for particle size distribution measurement, light microscopy, confocal laser scanning microscopy, electron microscopy, atomic force microscopy, static light scattering, laser diffractometry, dynamic light scattering, centrifugation, electric pulse measurement, chromatography, and ultrasonic attenuation are known, for example. An apparatus corresponding to each principle is commercially available.

In particle size measurement of the ceramide containing particles in the present invention, dynamic light scattering is preferably used because of a particle size range and easiness of measurement.

Examples of commercially available measuring apparatuses using dynamic light scattering include Nanotrac UPA (Nikkiso Co., Ltd.), Dynamic Light Scattering Particle Size Distribution Analyzer LB-550 (Horiba, Ltd.), and Fiber-Optics Particle Size Analyzer FPAR-1000 (Otsuka Electronics Co., Ltd.) Herein, an average particle size d50, a volume average particle size Mv, and a number average particle size Mn were determined using a Nanotrac UPA.

Assuming that there is a group of particles and the entire volume of the group of particles is 100%, a cumulative curve is determined. At this time, particle sizes at a point that the cumulative curve is 10%, 50%, and 90% are a 10% diameter, a 50% diameter, and a 90% diameter (μm), respectively. The 50% diameter is defined as the average particle size d50. Assuming that there is a group of particles, in the case that n1, n2, . . . ni, . . . nk particles respectively have a particle size d1, d2, . . . di, . . . dk in order of a smaller particle size, a surface area per particle is ai, and a volume ratio is vi, Mv is an average diameter determined from the following equation:

$$MV = \frac{V 1 \cdot d 1 + V 2 \cdot d 2 + … Vidi + … Vk \cdot dk}{V 1 + V 2 + … Vi + … Vk} \\ MV = \frac{\sum ( Vi \cdot di )}{\sum ( Vi )} = \frac{\sum ( Vi \cdot di )}{100} \qquad [ Equation 1 ]$$

Mn is an average diameter of the particles determined from imaginary distribution of the number of the particles all determined with a spherical calculation.

### [Other Oil Components]

The ceramide dispersed object of the present invention is composed as an oil phase by dispersing ceramide containing particles into an aqueous phase. An alternative form can be used such that an oil component different from ceramides such as the above-described natural ceramides (referred to as other oil component properly) and/or a solvent are contained in the oil phase so that oil droplet dispersed particles containing a natural ceramide in the oil component and/or the solvent exist as natural ceramide containing particles. In the case of using this form, the average particle size of the ceramide containing particles in the present invention means an average particle size of the oil droplet dispersed particles containing the ceramide containing particles.

### (2) A Fatty Acid Component

A fatty acid component in the present invention is at least one of (a) fatty acids having not less than 12 and not more than 20 carbon atoms, and (b) fatty acid salts. By such a fatty acid component, fine and stable ceramide containing particles containing ceramides can be obtained. This fatty acid component is not contained in a “surfactant” described later in the present invention.

As the (a) component, fatty acids having not less than 12 and not more than 20 carbon atoms can be used. Among them, fatty acids such as lauric acid, oleic acid, and isostearic acid having a solution form at normal temperature or at a temperature at the time of dispersion are more desirable. Examples of fatty acids having not less than 12 and not more than 20 carbon atoms include lauric acid, myristic acid, palmitic acid, oleic acid, stearic acid, isostearic acid, linoleic acid, α-linolenic acid, and γ-linolenic acid. The fatty acids of the (a) component are contained in the ceramide dispersed object as an oil phase component.

The fatty acid salts of the (b) component can be dissolved in an aqueous medium irrespective of the melting point of the fatty acids. Accordingly, from the viewpoint of solubility in the mixing step, the fatty acid component in the present invention may be composed of fatty acids having any melting point, and may be either of saturated fatty acids and unsaturated fatty acids. Examples of salts that compose the fatty acid salts include metal salts such as sodium and potassium, basic amino acid salts such as L-arginine, L-histidine, and L-lysine, and alkanolamine salts such as triethanolamine. A kind of the salts is properly selected according to a kind of the fatty acid used. From the viewpoint of solubility and stability of a dispersion liquid, metal salts such as sodium are preferable. The fatty acid salts of the (b) component are soluble in an aqueous medium, and therefore, can be used as an aqueous phase component for the ceramide dispersed object.

The fatty acid component in the ceramide dispersed object of the present invention may be fatty acids having not less than 12 and not more than 20 carbon atoms. Examples of the fatty acids having not less than 12 and not more than 20 carbon atoms can include lauric acid, myristic acid, palmitic acid, palmitoleic acid, stearic acid, oleic acid, 12-hydroxystearic acid, tolic acid, isostearic acid, linoleic acid, α-linolenic acid, γ-linolenic acid, and salts thereof. These can be used alone or in combination with not less than two kinds. Among them, from the viewpoint of having a solution form at normal temperature or a temperature at the time of dispersion, as the fatty acid component in the present invention, at least one kind selected from the group consisting of myristic acid, palmitic acid, palmitoleic acid, lauric acid, stearyl acid, isostearic acid, oleic acid, γ-linolenic acid, α-linolenic acid, linoleic acid, and salts thereof is preferable, and oleic acid is particularly preferable.

### (3) Polyhydric Alcohols

Polyhydric alcohols have a moisturizing function, a viscosity control function, and the like. Polyhydric alcohols also have a function to assist spreading of an interface by reducing boundary tension between water and fat and oil components and a function to assist formation of fine and stable particulates.

Then, it is preferable that polyhydric alcohols be contained in the ceramide dispersed object because the diameter of the dispersed particles of the ceramide dispersed object can be made finer, and can be stably held for a long period of time while the particle size is kept at the fine particle size.

Addition of polyhydric alcohols can reduce water activity of the ceramide dispersed object to suppress propagation of microorganisms.

As polyhydric alcohols that can be used for the present invention, alcohols except monohydric alcohol can be used without particular limitation.

Examples of polyhydric alcohols include glycerol, diglycerol, triglycerol, polyglycerol, 3-methyl-1,3-butanediol, 1,3-butylene glycol, isoprene glycol, polyethylene glycol, 1,2-pentanediol, 1,2-hexandiol, propylene glycol, dipropylene glycol, polypropylene glycol, ethylene glycol, diethylene glycol, pentaerythritol, neopentyl glycol, maltitol, reduced starch syrup, sucrose, lactitol, palatinit, erythritol, sorbitol, mannitol, xylitol, xylose, glucose, lactose, mannose, maltose, galactose, fructose, inositol, pentaerythritol, maltotriose, sorbitan, trehalose, amylolysis sugar, and amylolysis sugar reduced alcohol. These can be used alone or as a mixture in combination with several kinds.

### (4) Polysaccharide Fatty Acid Esters

The ceramide dispersed object of the present invention contains polysaccharide fatty acid esters. Usually, an emulsified and dispersed object as the ceramide dispersed object of the present invention may be formulated in a form of various kinds of organic salts or mineral salts in order to stably formulate the respective components having various functions. Increased organic salts or mineral salts are likely to cause the so-called salting-out, i.e., phenomena such as turbidity, condensation, precipitation, thickening, and separation. Particularly in the case of a formula in which transparency is important, such salting-out may impair transparency, and is not preferable. Because the ceramide dispersed object of the present invention contains polysaccharide fatty acid esters, the ceramide dispersed object of the present invention has dispersion stability, and additionally, can suppress the so-called salting-out favorably even in the case where various organic or mineral salts are blended.

It is also possible to improve stability over time of the dispersed object by suppressing precipitation of the ceramide dispersed object, for example.

In polysaccharide fatty acid esters in the present invention, a polysaccharide portion is composed of sugar units such as glucose or fructose having an average degree of polymerization of not less than 2 and not more than 140. Examples of polysaccharides can include disaccharides such as sucrose; and polysaccharides having not less than 6 saccharides such as oligosaccharides, inulins (approximately 2 to 60 fructoses are linearly bonded to glucose), starch, and dextrins. Dextrins, inulins, and a combination thereof are preferable, and inulins are more preferable in order to effectively suppress the salting-out phenomena such as turbidity, condensation, precipitation, thickening, and separation of the ceramide dispersed object caused by addition of salts. Inulins are an oligosaccharide mainly containing D-fructose as a basic component, and usually have approximately 2 to 60 flanoid fructose units that shows a structure having β-1,2-bonded flanoid fructose and α-D-glucose sucrose-bonded to a reducing end.

A fatty acid part that forms esters with polysaccharides is preferably fatty acids having not less than 12 and not more than 18 carbon atoms in order to effectively suppress precipitation caused by salts. Examples of the fatty acids can include caprylic acid, capric acid, lauric acid, myristic acid, palmitic acid, oleic acid, stearic acid, isostearic acid, linoleic acid, linolenic acid, ethylhexanoic acid, behenic acid, and behenic acid.

Examples of such polysaccharide fatty acid esters can include dextrin fatty acid esters, sucrose fatty acid esters, starch fatty acid esters, oligosaccharide fatty acid esters, and inulin fatty acid esters. Inulin fatty acid esters and dextrin fatty acid esters are preferable. Examples of inulin fatty acid esters can include octanoyl inulin, decanoyl inulin, lauroyl inulin, myristoyl inulin, lauryl carbamoyl inulin, palmitoyl inulin, stearoyl inulin, arachyl inulin, behenyl inulin, oleoyl inulin, 2-ethylhexanoyl inulin, isomyristoyl inulin, isopalmitoyl inulin, isostearoyl inulin, and isooleoyl inulin. As polysaccharide fatty acid esters of the present invention, stearoyl inulin, lauryl carbamoyl inulin, palmitoyl dextrin, palmitoyl/octanoyl dextrin, and myristoyl dextrin are suitable from the viewpoint of stability of the ceramide dispersed object. Lauryl carbamoyl inulin is most preferable.

Lauryl carbamoyl inulin has an HLB value of approximately 8, and has low solubility and good dispersibility for an oil component. Lauryl carbamoyl inulin also has low solubility in water, and aggregates in an aqueous phase. In an oil-in-water emulsion, lauryl carbamoyl inulin is located on a surface of the dispersed particles by hydration of an inulin skeleton to form a three-dimensional barrier. The three-dimensional barrier forms an emulsified structure such that the three-dimensional barrier surrounds the dispersed particles in an aqueous phase.

### (5) A Good Solvent for Ceramides

The ceramide dispersed object of the present invention may further contain a good solvent for ceramides. This good solvent is not included in the “oil component” herein.

The good solvent for ceramides may be a liquefied solvent at normal temperature that can dissolve at least not less than 0.1% by mass of ceramides at 25° C., for example. In the present invention, the good solvent may be any substance as long as it is oils and fats and solvents that dissolve not less than 0.1% by mass of ceramides.

The good solvent in the present invention is preferably a water soluble organic solvent.

The water soluble organic solvent in the present invention is used as an oil phase containing a natural component to be mixed with an aqueous solution described later. This aqueous organic solvent is also a principal component of an extraction solution that extracts a natural component. In other words, in the present invention, the natural component is used to be mixed with the aqueous solution in a state that the natural component is extracted to the extraction solution mainly containing the water soluble organic solvent.

The water soluble organic solvent used for the present invention means an organic solvent having not less than 10% by mass of solubility at 25° C. to water. From the viewpoint of stability of the finished dispersed object, the solubility in water is preferably not less than 30% by mass, and more preferably not less than 50% by mass.

The water soluble organic solvent may be used alone, or may be used as a mixed solvent in combination with several water soluble organic solvents. The water soluble organic solvent may also be used as a mixture with water. In the case of using a mixture of the water soluble organic solvent and water, the water soluble organic solvent contained is preferably at least not less than 50% by volume, and more preferably not less than 70% by volume.

The water soluble organic solvent is preferably used in order to mix an oil phase component to prepare an oil phase in the method for producing a ceramide dispersed object. The water soluble organic solvent is preferably removed after mixing with an aqueous phase.

Examples of such a water soluble organic solvent include methanol, ethanol, 1-propanol, 2-propanol, 2-butanol, acetone, tetrahydrofuran, acetonitrile, methyl ethyl ketone, dipropylene glycol monomethyl ether, methyl acetate, methyl acetoacetate, N-methylpyrrolidone, dimethylsulfoxide, ethylene glycol, 1,3-butanediol, 1,4-butanediol, propylene glycol, diethylene glycol, triethylene glycol, and mixtures thereof. Among these, in the case that application is limited to food products, ethanol, propylene glycol, or acetone is preferable, and ethanol or a mixed solution of ethanol and water is particularly preferable.

As mentioned above, the method for mixing fluids, the method for producing particulates, and the particulates according to the present invention have been described in detail. The present invention, however, is not limited to the above-mentioned embodiments, and various kinds of improvement and modifications may be made without deviating from the gist of the present invention.

### EXAMPLES

Hereinafter, specific examples according to the present invention will be shown to describe the present invention more in detail. Two kinds of fluids were mixed using the microreactor 30 shown in FIG. 4 to produce particulates.

Each component described in Composition of an oil phase solution 1 below was stirred for approximately 30 minutes at room temperature to prepare an oil phase solution 1. An aqueous phase solution 1 below was prepared as follows: lauryl carbamoyl inulin (INUTEC SP1: made by Nihon Siber Hegner K.K., the same in the followings) was added to pure water, heated to approximately 50° C., and fully stirred and dissolved; subsequently, the remaining components were added and mixed, and a temperature of the solution was adjusted at 30° C.

### <Composition of an Oil Phase Solution 1>

| | |
|---|---|
| Ceramide 3B [natural ceramide, Example 1-11] | 0.900 parts |
| Ceramide 6 [natural ceramide, Examples 1-7] | 1.100 parts |
| Oleic acid | 0.200 parts |
| Ethanol [water soluble organic solvent] | 97.800 parts |

### <Composition of an Aqueous Phase Solution 1>

| | |
|---|---|
| Pure water | 96.860 parts |
| Lauryl carbamoyl inulin | 0.290 parts |
| Glycerol | 1.430 parts |
| 1,3-butanediol | 1.430 parts |
| 0.1 mol sodium hydroxide | proper amount |
| (the final ceramide dispersed object at pH = 7.4) | |

The oil phase solution 1 (oil phase) and the aqueous phase solution 1 (aqueous phase) obtained were mixed with each other in a ratio (mass ratio) of 1:7 using the microreactor. Thus, the ceramide dispersion liquid 1 was obtained. Operating conditions on the microreactor is as follows.

### —A Ratio of a Flow Rate—

The oil phase and the aqueous phase were sent in a ratio of 1:7.

### Example 1

A first solution and a second solution were sent at a flow rate of 80 ml/min. to 960 ml/min. using the microreactor in which a width of a first passage was 100 μm, a depth thereof was 200 μm, and the number of the passages thereof was five; a width of a second passage was 700 μm, a depth thereof was 200 μm, and the number of the passages thereof was five; a merging section equivalent diameter φ was 1.34 mm and a depth of the merging section was 200 μm; an outlet diameter φ was 0.8 mm; and a mixing zone was 0.28 mm³. As a result, a dynamic pressure reached 199 kPa at 320 ml/min. (the first solution 40 ml/min.:the second solution 280 ml/min.=1:7, the total flow rate per second is 1.9×10⁴ times the volume of the mixing zone), a particle size d50=3.7 nm and distribution Mv/Mn=1.18. At this flow rate or more, the particle size d50 was not more than 5 nm, and the distribution Mv/Mn was not more than 1.3.

### Example 2

A first solution and a second solution were sent at a flow rate of 80 ml/min. to 400 ml/min. using the microreactor in which a width of a first passage was 150 μm, a depth thereof was 200 μm, and the number of the passages thereof was three; a width of a second passage was 600 μm, a depth thereof was 200 μm, and the number of the passages thereof was three; a merging section equivalent diameter φ was 0.8 mm and a depth of the merging section was 200 μm; an outlet diameter φ was 0.8 mm; and a mixing zone was 0.10 mm³. As a result, a dynamic pressure reached 124 kPa at 200 ml/min. (the first solution 25 ml/min.:the second solution 175 ml/min.=1:7, the total flow rate per second is 3.3×10⁴ times the volume of the mixing zone), a particle size d50=4.7 nm and distribution Mv/Mn=1.29. At this flow rate or more, the particle size d50 was not more than 5 nm, and the distribution Mv/Mn was not more than 1.3.

### Example 3

A first solution and a second solution were sent at a flow rate of 80 ml/min. to 400 ml/min. using the microreactor in which a width of a first passage was 100 μm, a depth thereof was 400 μm, and the number of the passages thereof was three; a width of a second passage was 400 μm, a depth thereof was 400 μm, and the number of the passages thereof was three; a merging section equivalent diameter φ was 0.8 mm and a depth of the merging section was 400 μm; an outlet diameter φ was 0.8 mm; and a mixing zone was 0.20 mm³. As a result, a dynamic pressure reached 100 kPa at 240 ml/min. (the first solution 30 ml/min.:the second solution 210 ml/min.=1:7, the total flow rate per second is 2.0×10⁴ times the volume of the mixing zone), a particle size d50=3.1 nm and distribution Mv/Mn=1.23. At this flow rate or more, the particle size d50 was not more than 5 nm, and the distribution Mv/Mn was not more than 1.3.

### Example 4

A first solution and a second solution were sent at a flow rate of 160 ml/min. to 480 ml/min. using the microreactor in which a width of a first passage was 400 μm, a depth thereof was 400 μm, and the number of the passages thereof was three; a width of a second passage was 400 μm, a depth thereof was 400 μm, and the number of the passages thereof was three; a merging section equivalent diameter φ was 0.8 mm and a depth of the merging section was 400 μm; an outlet diameter φ was 0.8 mm; and a mixing zone was 0.20 mm³. As a result, a dynamic pressure reached 144 kPa at 320 ml/min. (the first solution 40 ml/min.:the second solution 280 ml/min.=1:7, the total flow rate per second is 2.7×10⁴ times the volume of the mixing zone), a particle size d50=3.9 nm and distribution Mv/Mn=1.26. At this flow rate or more, the particle size d50 was not more than 5 nm, and the distribution Mv/Mn was not more than 1.3.

### Example 5

A first solution and a second solution were sent at a flow rate of 40 ml/min. to 120 ml/min. using the microreactor in which a width of a first passage was 200 μm, a depth thereof was 200 μm, and the number of the passages thereof was three; a width of a second passage was 200 μm, a depth thereof was 200 μm, and the number of the passages thereof was three; a merging section equivalent diameter φ was 0.4 mm and a depth of the merging section was 200 μm; an outlet diameter φ was 0.4 mm; and a mixing zone was 0.025 mm³. As a result, a dynamic pressure reached 144 kPa at 80 ml/min. (the first solution 10 ml/min.:the second solution 70 ml/min.=1:7, the total flow rate per second is 5.3×10⁴ times the volume of the mixing zone), a particle size d50=3.7 nm and distribution Mv/Mn=1.21. At this flow rate or more, the particle size d50 was not more than 5 nm, and the distribution Mv/Mn was not more than 1.3.

### Example 6

A first solution and a second solution were sent at a flow rate of 8 ml/min. to 40 ml/min. using the microreactor in which a width of a first passage was 100 μm, a depth thereof was 100 μm, and the number of the passages thereof was five; a width of a second passage was 100 μm, a depth thereof was 100 μm, and the number of the passages thereof was five; a merging section equivalent diameter φ was 0.34 mm and a depth of the merging section was 100 μm, an outlet diameter φ was 0.34 mm; and a mixing zone was 0.009 mm³. As a result, a dynamic pressure reached 124 kPa at 24 ml/min (the first solution 3 ml/min.:the second solution 21 ml/min.=1:7, the total flow rate per second is 4.4×10⁴ times the volume of the mixing zone), a particle size d50=3.8 nm and distribution Mv/Mn=1.26. At this flow rate or more, the particle size d50 was not more than 5 nm, and the distribution Mv/Mn was not more than 1.3.

### Example 7

A first solution and a second solution were sent at a flow rate of 20 ml/min. to 200 ml/min. using a T-shaped reactor in which a diameter of a first passage was 300 μm and the number of the passages thereof was one; a diameter of a second passage was 300 μm and the number of the passages thereof was one; and a mixing zone was 0.023 mm³. As a result, a dynamic pressure reached 138 kPa at 80 ml/min. (the first solution 10 ml/min.:the second solution 70 ml/min.=1:7, the total flow rate per second is 5.6×10⁴ times the volume of the mixing zone), a particle size d50=3.9 nm and distribution Mv/Mn=1.19. At this flow rate or more, the particle size d50 was not more than 5 nm, and the distribution Mv/Mn was not more than 1.3.

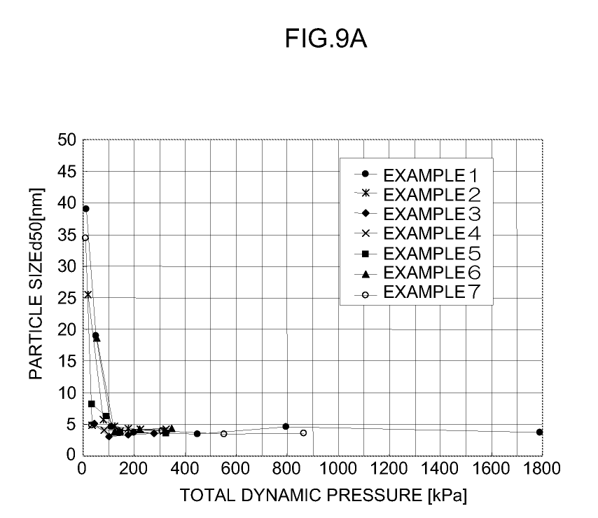

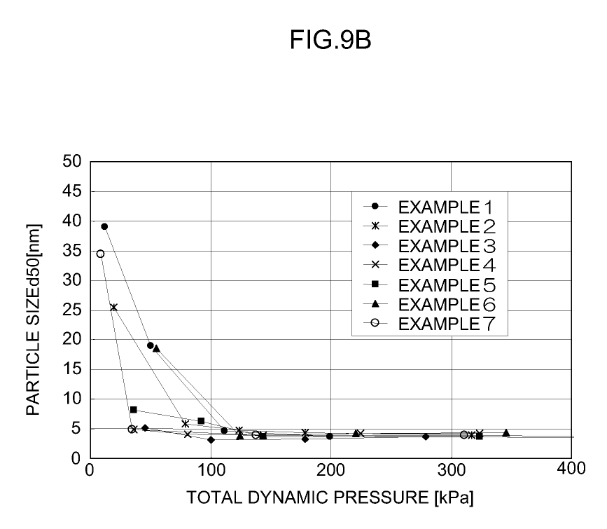

FIGS. 9A and 9B respectively show a graph showing a relationship between the total dynamic pressure (abscissa) and the particle size d50 (ordinate) with respect to Examples 1 to 7. In FIG. 9A, at the maximum value of the total dynamic pressure of 1800 kPa, the particle size d50 of Examples 1 to 7 was plotted. FIG. 9B is a graph shown where the maximum value of the abscissa in FIG. 9A is 400 kPa.

As shown in FIG. 9, when the total dynamic pressure is from 10 to approximately 100 kPa, the particle size d50 changes according to the total dynamic pressure. In other words, the particle size d50 can be controlled by controlling the total dynamic pressure.

On the other hand, when the total dynamic pressure exceeds approximately 100 kPa, the particle size d50 is approximately 5 nm regardless of the total dynamic pressure. In order to obtain the particulates having the particle size d50 of approximately 5 nm, it is desirable that the total dynamic pressure exceed approximately 100 kPa. In this case, the total dynamic pressure is desirably a value close to 100 kPa. This is because a larger total dynamic pressure leads to more pressure loss so that energy is consumed more than needed.

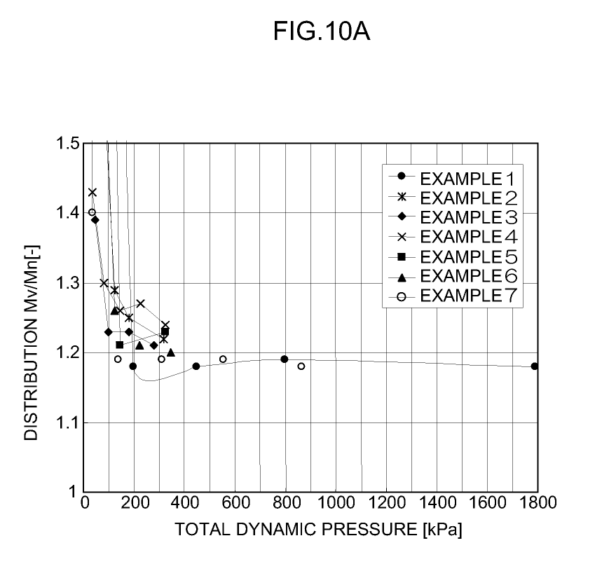

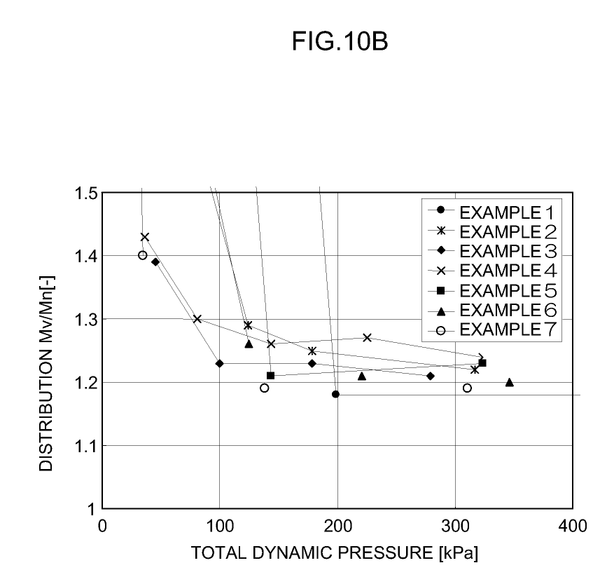

FIGS. 10A and 10B respectively show a graph showing a relationship between the total dynamic pressure (abscissa) and the distribution Mv/Mn (ordinate) with respect to Examples 1 to 7. In FIG. 10A, at the maximum value of the total dynamic pressure of 1800 kPa, the distribution Mv/Mn of Examples 1 to 7 was plotted. FIG. 10B is a graph shown where the maximum value of the abscissa in FIG. 10A is 400 kPa. The distribution Mv/Mn (volume average particle size/number average particle size) is an index indicating monodispersity, which shows that monodispersity is higher at a value closer to 1.

As shown in FIG. 10, when the total dynamic pressure is from 0 to approximately 200 kPa, the distribution Mv/Mn changes according to the total dynamic pressure. In other words, the distribution Mv/Mn can be controlled by controlling the total dynamic pressure.

On the other hand, when the total dynamic pressure exceeds approximately 200 kPa, the distribution Mv/Mn is approximately 1.2 regardless of the total dynamic pressure. In order to obtain particulates having the distribution Mv/Mn of approximately 1.2, it is desirable that the total dynamic pressure exceed approximately 200 kPa. In this case, the total dynamic pressure is desirably a value close to 150 kPa or a value close to 200 kPa (desirable value varies with Examples). This is because a larger total dynamic pressure leads to more pressure loss so that energy is consumed more than needed.

What is claimed is:

1. A method for mixing at least two kinds of fluids in a microreactor, comprising the steps of:
supplying a first fluid to a mixing zone through a first passage;
supplying a second fluid to the mixing zone through a second passage;
controlling a total value of dynamic pressures of the first and second fluids and merging the first and second fluids in the mixing zone; and
flowing the first and second fluids mixed in the mixing zone into a third passage.

2. The method for mixing fluids according to claim 1, wherein the total value of the dynamic pressures of the first and second fluids is not less than 100 kPa.

3. The method for mixing fluids according to claim 2, wherein the total value of the dynamic pressures of the first and second fluids is not less than 200 kPa.

4. The method for mixing fluids according to claim 1, wherein a volume of the mixing zone is 0.001 mm³ to 1 mm³.

5. The method for mixing fluids according to claim 2, wherein a volume of the mixing zone is 0.001 mm³ to 1 mm³.

6. The method for mixing fluids according to claim 3, wherein a volume of the mixing zone is 0.001 mm³ to 1 mm³.

7. The method for mixing fluids according to claim 1, wherein a total flow rate per second of the fluid supplied to the mixing zone is 1×10³ to 1×10⁵ times the volume of the mixing zone.

8. The method for mixing fluids according to claim 5, wherein a total flow rate per second of the fluid supplied to the mixing zone is 1×10³ to 1×10⁵ times the volume of the mixing zone.

9. The method for mixing fluids according to claim 6, wherein a total flow rate per second of the fluid supplied to the mixing zone is 1×10³ to 1×10⁵ times the volume of the mixing zone.

10. The method for mixing fluids according to claim 1, wherein at least one passage of the first passage and the second passage includes a plurality of subpassages branched halfway to the mixing zone, and a central axis of one fluid that flows through at least one subpassage of the plurality of subpassages intersects with a central axis of other fluid that flows through other passage at one point in the mixing zone.

11. The method for mixing fluids according to claim 8, wherein at least one passage of the first passage and the second passage includes a plurality of subpassages branched halfway to the mixing zone, and a central axis of one fluid that flows through at least one subpassage of the plurality of subpassages intersects with a central axis of other fluid that flows through other passage at one point in the mixing zone.

12. The method for mixing fluids according to claim 9, wherein at least one passage of the first passage and the second passage includes a plurality of subpassages branched halfway to the mixing zone, and a central axis of one fluid that flows through at least one subpassage of the plurality of subpassages intersects with a central axis of other fluid that flows through other passage at one point in the mixing zone.

13. The method for mixing fluids according to claim 1, wherein a diameter of the third passage is not more than an equivalent diameter of a merging passage.

14. The method for mixing fluids according to claim 11, wherein a diameter of the third passage is not more than an equivalent diameter of a merging passage.

15. The method for mixing fluids according to claim 12, wherein a diameter of the third passage is not more than an equivalent diameter of a merging passage.

16. A method for producing particulates comprising:
mixing a first fluid with a second fluid according to the method for mixing fluids according to f claim 1, the first fluid being a solution obtained by dissolving an organic compound in a good solvent having relatively high solubility for the organic compound, and the second fluid being a solution obtained by dissolving the organic compound in a poor solvent having relatively low solubility for the organic compound.

17. The method for producing particulates according to claim 16 wherein the organic compound is a material insoluble or sparingly soluble in an aqueous medium.

18. Particulates produced by the method for producing particulates according to claim 16.

19. Particulates produced by the method for producing particulates according to claim 17.
